(19) United States
(12) Patent Application Publication
(10) Pub. No.: US 2007/0023512 A1
(43) Pub. Date: Feb. 1, 2007

(54) INVENTORY MANAGEMENT SYSTEM USING RFID TAGS TO AID IN DISPENSING AND RESTOCKING INVENTORY

(75) Inventors: Richard Miller, Pittsburgh, PA (US); Manoj Wangu, Wexford, PA (US); Bruce Thompson, Pittsburgh, PA (US); Patrick Braun, Pittsburgh, PA (US); Russell Gadagno, Harrison City, PA (US); Robert Meek, Pittsburgh, PA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: MCKESSON AUTOMATION INC., Cranberry, PA (US)

(21) Appl. No.: 11/423,326

(22) Filed: Jun. 9, 2006

Related U.S. Application Data
(60) Provisional application No. 60/689,508, filed on Jun. 10, 2005.

(51) Int. Cl. G06Q 30/00

(52) U.S. Cl. 235/385

(57) ABSTRACT

An inventory management system is provided that utilizes radio frequency identification (RFID) technology to drive RFID tags to provide a notification signal, such as by illuminating light emitting diodes (LEDs) embodied by the RFID tags. The RFID tags may be placed on medication bins within a pharmacy pick-station in order to indicate which medication to select for processing an order or in which medication bin to place a particular medication being restocked. A method and computer program product for managing inventory, as well as an automated dispensing cabinet are further provided.

### CROSS-REFERENCE TO RELATED APPLICATIONS

The present application claims priority from U.S. Provisional Application No. 60/689,508 filed Jun. 10, 2005 and entitled “Inventory Management System Using RFID Tags to Aid in Dispensing and Restocking Inventory,” the contents of which are hereby incorporated herein by reference in their entirety.

### BACKGROUND OF THE INVENTION

1. Field of the Invention

Exemplary embodiments of the present invention are directed toward inventory management systems and, more particularly, to inventory management systems which use radio frequency identification (RFID) tags to aid in dispensing and restocking of inventory.

2. Description of the Background

Medical facilities, such as hospitals, nursing homes, and the like, have a centralized location such as a pharmacy department or materials management department within the facility to coordinate the dispensing of drugs or medical supplies to the patients of the medical facility. The departments utilizing medications and medical supplies in such facilities have long been burdened with the increasingly complex record keeping and inventory management that results from caring for hundreds, if not thousands, of patients every day. Various methods have been employed to assist a centralized pharmacy or other centralized medical supply departments with maintaining accurate records while attempting to reduce the burden of managing all of the information associated with the distribution of medications and medical supplies. The responsibilities of the centralized supply include: filling individual patient prescriptions on a daily basis; administering drugs using the five rights: right drug, right patient, right dose, right time and right route, dispensing medical supplies to patients; maintaining sufficient inventory of each drug or medical supply so as to have sufficient quantities on hand to administer to patients on a daily basis; tracking of drug interactions to prevent a patient from being given a drug that has adverse affects when combined with other drugs; accounting for the purchase of medications and medical supplies for use in the facility; accounting associated with dispensing of medications and medical supplies to individual patients; tracking of medication expiration dates to rid inventories of expired medications; and tracking of drug lot numbers, for example, in the event of a recall of a particular drug or drug lot number.

Medical facilities will dispense medications in one of three modes: centralized, decentralized, or a hybrid of partial decentralization. In facilities that are partly or fully decentralized, a very important function of the centralized pharmacy or materials management department is to restock various inventory locations, e.g. nurses stations, unit-based cabinets, satellite pharmacies, or off-site facilities in a network, with the quantity and types of medications and medical supplies that must be dispensed by the decentralized locations on a daily basis.

Replenishing the inventory in a centralized hospital-wide nursing unit-based cabinet (UBC) system is a time consuming, laborious, and often awkward process for pharmacy departments. The typical manual pick process using paper-based replenishment reports is certainly not optimal. In the typical manual pick process, a hard copy report is generated manually. The pharmacy technician or worker responsible for filling the orders works from the hard copy report to pick the requested items. This process is time consuming and complex. Patient bar-code labels are generally not used. Additionally, the manual pick process requires significant staff training and practice as well as inventory memorization. The accuracy and safety of the picks are ensured by a manual check of the medications against the dispensing list. Additionally, restocking and billing requires the manual count of medications and completion of paperwork. Finally, medication inventory management is performed by manual, time-consuming counts and replenishments must be ordered manually. Thus, pharmacy departments are very interested in a better way of managing their current UBC replenishment processes.

Automated systems for dispensing and restocking inventory are currently used in pharmacies in place of the manual pick process. For example, “pick-to-light” technology or a “place-to-light” may be used. Both of these systems involve the use of specially designed, hardwired workstations and pharmacy units. While these systems offer substantial advantages over the manual pick process, some facilities may not have the resources to convert to such an automated system and basically reengineer the whole pharmacy. Therefore, the need exists for an automated inventory management system that improves workflow, safety and cost efficiency to hospital pharmacies of any size volume or staffing level.

### BRIEF SUMMARY OF THE INVENTION

The present disclosure utilizes Radio Frequency Identification (“RFID”) technology to drive the illumination of RFID tags placed on medication storage locations within a pharmacy pick-station to indicate the medication to select for the order being processed. The present disclosure may further cause the quantity of the particular medication, or supply, to be displayed on the tag using, for example, a liquid crystal display (LCD). The present disclosure also directs the user to scan the bar code, or similar identification code, on the medication, or other supply, to validate that the proper medication and quantity was accurately selected. Error messages are provided should the incorrect medication (or supply) or quantity be selected for the order being processed. An error message or alert may similarly be provided where the medication has expired, or is about to expire. Additionally, the present disclosure tracks the perpetual inventory for each medication storage location and will automatically generate electronic reorders for submission to the distribution system for replenishment. The present disclosure may further track and generate periodic reports of the expiration dates of each medication maintained in the medication storage locations.

The present disclosure is a system to enhance medication safety through RFID-enabled guiding light technology. The system allows pharmacies to continue to use their existing medication-shelving units. The system includes RFID tags that easily attach to existing medication bins as well as antennas that communicate with the RFID tags and the system's workflow software. The system includes workflow software, such as the Connect-Rx® software, available from McKesson Automation Inc., Cranberry Township, Pa., U.S.A., that integrates with any pharmacy information system to enhance profile dispensing and provide online inventory management. RFID-enabled guiding light technology directs technicians to quickly and accurately select the correct medication and dose from the pick list. Additionally, the present disclosure uses bar-code scanning to double check accuracy and help automate accounting of returned medications.

The present disclosure enables hospital pharmacies to streamline dispensing of medications and optimize existing pharmacy resources. The present disclosure simplifies the crediting of returned medications and improves pharmacy inventory management.

The present disclosure interfaces directly with pharmacy information system to obtain the pick list. While the present disclosure is paperless, a paper-based backup system may also be provided in the event that the system becomes non-operational. Providing a paper-based backup system provides not only redundancy within the system but also the ability to provide hard copy reports for any required state and/or local regulations. The system includes a computer terminal with workflow software, such as the Connect-Rx® software, to provide onscreen guidance to the technician. The system includes guiding light technology that streamlines profile dispensing processes. Bar code verification ensures right medication, right dosage and right quantity. Further the system automatically generates patient bar-code labels at the time of dispensing. The system correlates patients with medications to enhance patient safety and automatically tracks inventory.

The present disclosure decreases the time required to train technicians through automation and RFID-enabled guiding light technology.

The present disclosure includes bar-code scanning to permit quick credit processing for unused medications returned to the pharmacy. Additionally, the workflow software, such as the Connect-Rx® software, automatically updates billing and inventory.

The present disclosure allows for perpetual inventory assurance through online tracking and management as well as automated electronic reordering direct to the wholesaler using additional workflow software such as the FulFill-Rx^{SM} software, available from McKesson Automation Inc., Cranberry Township, Pa., U.S.A.

In accordance with one aspect, a method of managing inventory is provided. In one exemplary embodiment, the method includes: (1) receiving an indication of a medication; and (2) generating and transmitting a signal to a radio frequency identification (RFID) tag associated with a medication bin configured to house the medication, wherein, upon receiving the signal, the RFID tag is configured to generate a notification signal.

According to another aspect, a method is provided for prioritizing a plurality of antennas with respect to at least one of a plurality of radio frequency identification (RFID) tags. In one exemplary embodiment, the method includes: (1) associating the plurality of RFID tags with a corresponding plurality of medication bins, said plurality of medication bins configured to respectively house a corresponding plurality of medications; (2) determining a signal strength associated with respective antennas of the plurality of antennas with respect to at least one of the plurality of RFID tags; and (3) maintaining an ordering of the plurality of antennas for each RFID tag, wherein the ordering corresponds to the signal strength of respective antennas for the respective RFID tag.

In accordance with yet another aspect, an inventory management system is provided. In one exemplary embodiment, the system includes a plurality of medication bins configured to respectively house a corresponding plurality of medications. The system of this exemplary embodiment may further include a plurality of radio frequency identification (RFID) tags associated with the plurality of medication bins, wherein respective RFID tags are associated with a unique identification corresponding to the medication housed by the associated medication bin, and wherein respective RFID tags further comprise a notification device. The system of this exemplary embodiment may further include a software application adapted to operate on a processing element and configured to generate and transmit a signal to at least one of the plurality of RFID tags, wherein, upon receiving the signal, the RFID tag is configured to activate the corresponding notification device.

According to another aspect, a shelving unit is provided. In one exemplary embodiment, the shelving unit includes a plurality of medication bins configured to respectively house a corresponding plurality of medications, and a plurality of radio frequency identification (RFID) tags associated with the plurality of medication bins. Respective RFID tags of this exemplary embodiment may be associated with a unique identification corresponding to the medication housed by the associated medication bin. Respective RFID tags of this exemplary embodiment may further comprise a notification device, wherein respective RFID tags are configured to receive a signal and, upon receipt of the signal, activate the corresponding notification device.

According to one aspect, a computer program product for managing inventory is provided. The computer program product contains at least one computer-readable storage medium having computer-readable program code portions stored therein. The computer-readable program code portions of one exemplary embodiment include: (1) a first executable portion for receiving an indication of a medication; and (2) a second executable portion for generating and transmitting a signal to a radio frequency identification (RFID) tag associated with a medication bin configured to house the medication, wherein, upon receiving the signal, the RFID tag is configured to generate a notification signal.

### BRIEF DESCRIPTION OF THE SEVERAL VIEWS OF THE DRAWINGS

For the present disclosure to be easily understood and readily practiced, the present disclosure will now be described, for purposes of illustration and not limitation, in conjunction with the following figures, wherein:

FIG. 1 is one example of a pharmacy pick-station or workstation utilizing the present disclosure;

FIG. 2 is a flow chart illustrating the steps of a software application for interrogation of the antennas upon start-up;

FIG. 3 is a flow chart illustrating the steps for processing fill orders; and

FIG. 4 is a flow chart illustrating the steps for restocking of medications.

### DETAILED DESCRIPTION OF THE INVENTION

The present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

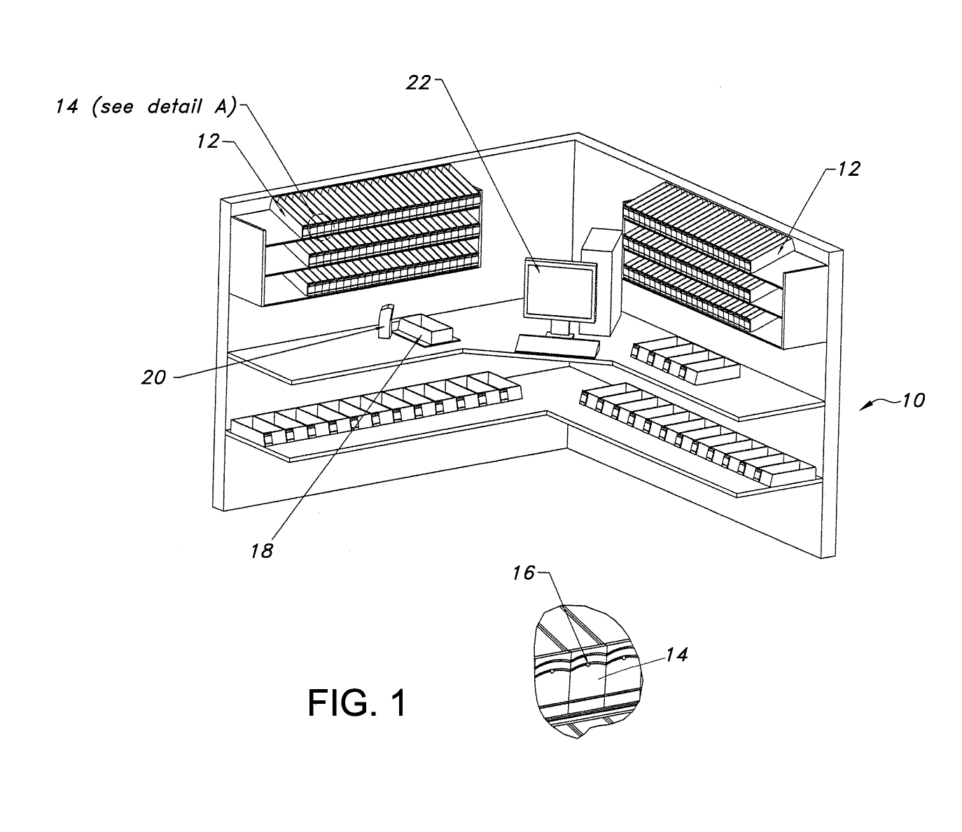

FIG. 1 is a diagram illustrating one example of a pharmacy pick-station utilizing the present disclosure. Those of ordinary skill in the art will recognize that the present invention may be utilized in various other workstation configurations. The workstation 10 includes medication storage locations or medication bins 12. Each medication bin 12 is capable of housing a particular medication or supply. “Medication” is used herein in its broadest sense to mean not only all types of pills, gelcaps and the like, but creams or ointments dispensed in tubes, patches, among others.

An RFID tag 14 containing an LED light 16 (see detail A) is placed on each medication bin 12. RFID tag 14 may also contain a barcode (not shown). An example of one type of RFID tag that may be used is the i-D2 tags, available from Identec Solutions, Inc., Kelowna, B.C. The RFID tags 14 are not hard-wired, and therefore, they can be placed anywhere, including on the existing pharmacy medication shelving-units. Each RFID tag 14 is capable of being associated with a unique identification number associated with the medication housed in the corresponding medication bin 12, such as the National Drug Code (“NDC”) number or Drug Identification Number (“DIN”) of the medication. The software application used with the system, which includes one or more computer program instructions stored in a memory and executed by a processing element, such as a computing device, may store an IP address associated with each RFID tag 14 and NDC number of the medication housed in the corresponding medication bin 12. Each RFID tag may additionally include a bar code associated with the NDC number of the medication housed in the corresponding medication bin 12. In addition, each RFID tag 14 may include a liquid crystal display (LCD), which may be used, for example, to display identification information associated with the medication housed in the corresponding medication bin, as well as a quantity of the medication that should be selected in order to fill a particular order being processed. Each workstation 10 may also include patient bins 18 for depositing all of the medication required to fill a particular patient order, a barcode scanner 20 and a workflow display or computer monitor 22. The system additionally includes one or more antennas (not shown) for communication between the software and the RFID tags. The antennas may be flat ceiling mounted antennas (i.e., patch antennas), whip antennas, or other similar antennas, located on the ceiling above the workstation 10. Alternatively, one or more rotatable antennas may be used. The rotatable antennas may be physically rotatable, have rotational waveguides attached thereto, or be electronically actuated in multiple planes.

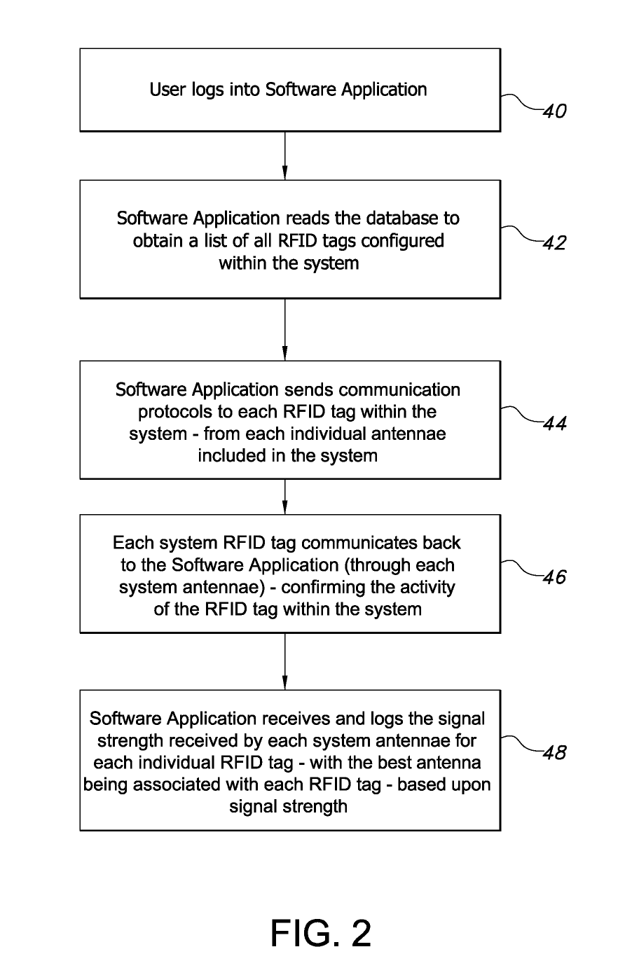

FIG. 2 illustrates the steps of a software application for interrogation of the antennas upon, for example, start-up. This process allows for the software, upon start up or at other times such as when inventory is moved, to interrogate each of the antennas to determine which antenna has the strongest signal connection with each RFID tag. Oftentimes obstructions such as cabinets, people, carts, etc. can block the signal between an antenna and an RFID tag. Upon start-up, the system interrogates the antennas to measure the signal strength of each antenna to each RFID tag. The system stores this information. That is, for each RFID tag the system software maintains a list of antennas according to signal strength. The antenna with the strongest signal strength to a particular RFID tag is referred to as the “best antenna” for that particular RFID tag. When an order is selected for fill, the software application communicates with the “best antenna”, i.e. that antenna having the strongest signal strength with the RFID tag associated with that particular medication. If the antenna from that signal fails to communicate with the RFID tag, the software application communicates with the antenna having the next strongest signal strength with the RFID tag associated with that particular medication, and so on, until communication occurs.

The interrogation process starts at 40 when a user logs into the software application. Upon log-in, the software application reads the database at 42 to obtain a list of all RFID tags configured within the system. The software application sends at 44 communication protocols to each RFID tag within the system from each individual antenna included in the system. At 46, each system RFID tag communicates back to the software application through each system antenna confirming the activity of the RFID tag within the system. The software application receives and logs at 48 the signal strength received by each system antenna for each individual RFID tag, with the best antenna being associated to each RFID tag based upon signal strength.

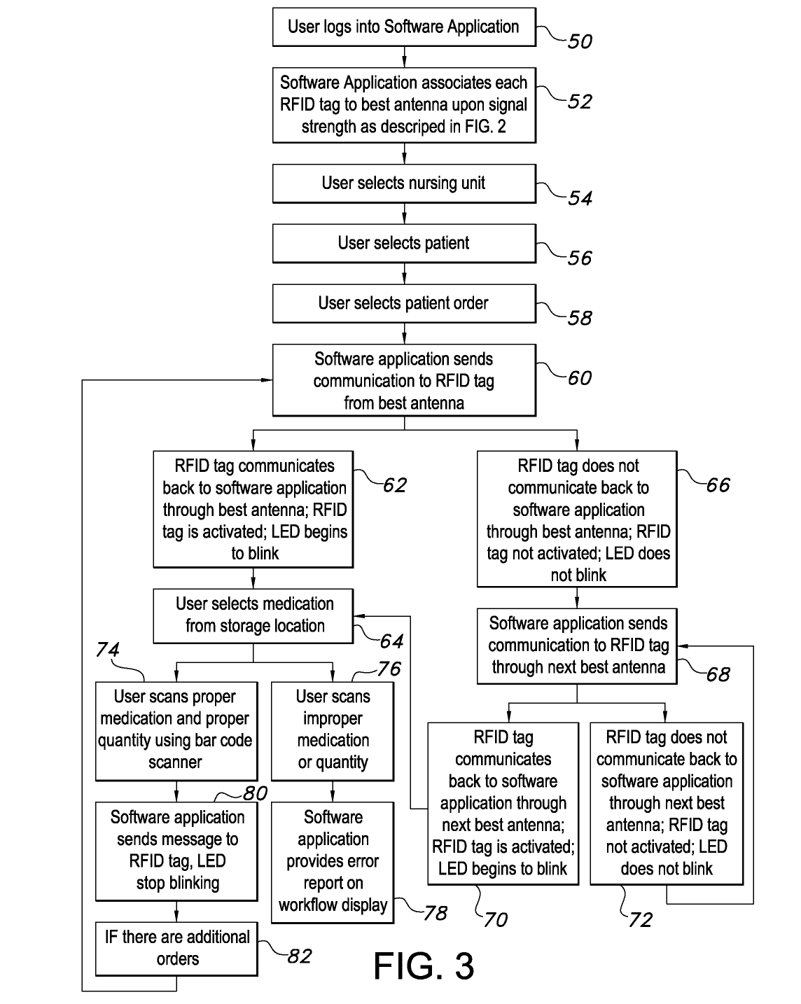

FIG. 3 is a flow chart illustrating the steps for processing fill orders. At 50, the user logs into the software application. The software application at 52 associates each RFID tag to the best antenna based upon signal strength as described in connection with FIG. 2. The user then selects at 54 a nursing unit to open for processing. The nursing unit displayed identifies a list of all patients for that nursing unit with patient orders to be filled. The user then selects at 56 a patient from the opened nursing unit list. In one exemplary embodiment, upon selecting a nursing unit to open for processing, the barcode labels associated with each patient corresponding with that unit are printed. In this exemplary embodiment, selecting a patient with patient orders to be filled comprises scanning the barcode label associated with that patient. Once selected, the patient displayed identifies a list of all patient orders (i.e., medications) to process. The user then selects at 58 a patient order from the selected patient's list of orders to process. In one exemplary embodiment, the system automatically selects the first patient order associated with the selected patient to be processed. Regardless of how it is selected, as an order is selected for processing, the software application will communicate at 60 to the appropriate RFID tag through the best antenna for that RFID tag. The RFID tag associated with the selected medication for the active order at 62 communicates back to the software application through the best antenna to acknowledge communication. At the same time, the RFID tag is activated and the embedded LED on the RFID tag begins to blink, thus directing the user to the proper medication storage location. In one exemplary embodiment, the LCD also associated with the RFID tag may display the quantity of the medication that should be selected in order to fill the order being processed. The user at 64 may then select medication from the storage location associated with the blinking LED. The RFID tags may employ notification schemes other than a blinking LED, if desired.

If the software application does not receive acknowledgement from the selected RFID tag at 66, the software application will attempt to communicate at 68 with the RFID tag through the next best antenna based upon signal strength determined during the system activation/interrogation. The RFID tag associated with the selected medication for the active order at 70 communicates back to the software application through the next best antenna to acknowledge communication. Once the RFID tag is activated and the embedded LED begins to blink, the user at 64 selects the medication from the storage location associated with the blinking RFID tag. If the software application does not receive acknowledgement from the selected RFID tag at 72, the software application will attempt to communicate at 68 with the RFID tag through the next best antenna.

Once the user at 64 selects medication from the storage location, the user preferably scans at 74 the proper medication and proper quantity into the software application utilizing a barcode scanner. Such information may be manually entered if the selected medication is not barcoded. If the user scans at 76 the incorrect medication or quantity for the active order, the software application will provide an error warning at 78 to the user on the workflow display or computer monitor, indicating that the incorrect medication or quantity was selected and allow the user to correct the order. Once the user scans at 74 the appropriate quantity of appropriate medication for the active order, the software application at 80 will send a message to the current RFID tag, through the best antenna, indicating that the current RFID tag can discontinue blinking the RFID tag LED. If at 82 there are additional orders within the selected patients' profile, the system application will then communicate to the next RFID tag utilizing the same process above, until all of the patient's orders are completed.

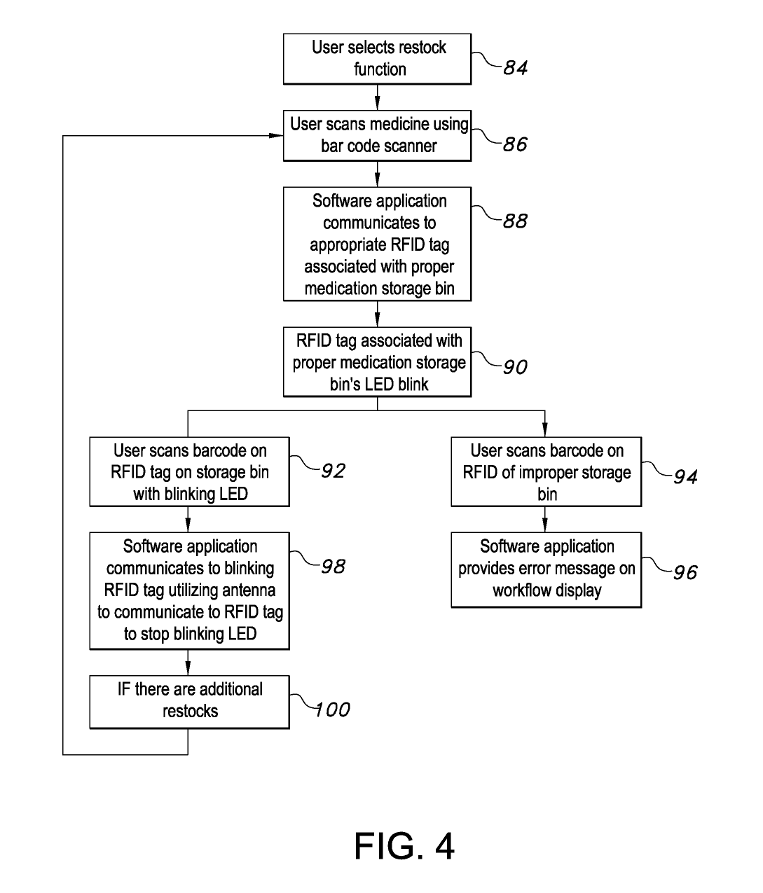

FIG. 4 is a flow chart illustrating the steps for restocking of medications. The user at 84 selects the restock function from the software application. The user at 86 then scans the medication to restock utilizing the systems barcode scanner. The software application at 88 communicates to the appropriate RFID tag associated with the proper medication storage location for the scanned medication utilizing the best antenna. The RFID tag associated with the proper medication storage location at 90 will begin to blink the embedded LED tag. The user may then place the medication in the storage location identified by the blinking LED tag. The user at 92 may scan the barcode associated with the blinking RFID tag, indicating that the medication was placed into the proper storage location. If the user at 94 scans the barcode on a RFID tag of an improper storage bin, the software application at 96 will provide an error message if an incorrect medication storage location is scanned for the selected medication to restock. Once the proper RFID tag is scanned, the software application at 98 will communicate to the blinking RFID tag, utilizing the best antenna to indicate to the RFID tag to discontinue blinking the RFID tag LED. The user at 100 will continue with the above process until all medications are restocked.

While the present disclosure has been described in connection with exemplary embodiments thereof, those of ordinary skill in the art will recognize that any modifications and variations are possible. Accordingly, the scope of the present disclosure is intended to be limited only by the following claims and to any equivalents thereof.

That which is claimed:

1. A method of managing inventory, said method comprising:
receiving an indication of a medication; and
generating and transmitting a signal to a radio frequency identification (RFID) tag associated with a medication bin configured to house the medication, wherein, upon receiving the signal, the RFID tag is configured to generate a notification signal.

2. The method of claim 1, wherein, in order to generate a notification signal, the RFID tag is configured to activate a light emitting diode (LED).

3. The method of claim 2, wherein activating the LED comprises causing the LED to at least one of continuously illuminate or blink.

4. The method of claim 1, wherein, in order to generate a notification signal, the RFID tag is configured to display a quantity associated with the medication on a liquid crystal display (LCD).

5. The method of claim 1, wherein receiving an indication of a medication comprises receiving a selection of an order to process, wherein the order comprises a medication to be dispensed.

6. The method of claim 1, wherein receiving an indication of a mediation comprises receiving an indication of a medication to be restocked.

7. The method of claim 6, further comprising reading a barcode associated with the medication to be restocked, which barcode corresponds to an identification number which serves as the indication of the medication.

8. The method of claim 1, wherein transmitting a signal to a radio frequency identification (RF ID) tag comprises transmitting the signal via at least one of a plurality of antennas.

9. The method of claim 8 further comprising:
determining via which of the plurality of antennas to transmit the signal.

10. The method of claim 9, wherein determining via which of the plurality of antennas to transmit the signal comprises:
transmitting an inquiry to the RFID tag via each of the plurality of antennas;
receiving, in response, a confirmation signal from the RFID tag via at least some of the plurality of antennas; and
determining a signal strength associated with each of the plurality of antennas, based at least in part on a signal strength associated with each of the confirmation signals, wherein the antenna having the strongest signal strength comprises the antenna via which the signal is transmitted.

11. The method of claim 10 further comprising:
maintaining an ordering of the plurality of antennas for each RFID tag, wherein the ordering corresponds to the signal strength associated with respective antennas for the respective RFID tag.

12. The method of claim 9, wherein the step of determining via which of the plurality of antennas to transmit the signal is repeated for each of a plurality of RFID tags corresponding with a plurality of medication bins.

13. The method of claim 8 further comprising:
receiving, in response to transmitting the signal, an acknowledgment from the RFID tag via the antenna via which the signal was transmitted to the RFID tag.

14. The method of claim 8 further comprising:
repeating transmission of the signal via a different one of the plurality of antennas, where an acknowledgement is not received from the RFID tag in response to a prior transmission of the signal.

15. The method of claim 5 further comprising:
receiving an identification of a medication retrieved from a medication bin;
comparing the identification with the medication included in the order received to process; and
generating an error message where the identification does not match the medication included in the order received.

16. The method of claim 15, further comprising reading a barcode associated with the medication retrieved from the medication bin, which barcode corresponds to an identification number which serves as the indication of the medication.

17. The method of claim 6 further comprising:
receiving an identification of a medication bin;
determining whether the identification matches the medication bin configured to house the medication to be restocked; and
generating an error message where the identification does not match the medication bin configured to house the medication to be restocked.

18. The method of claim 17, further comprising reading a barcode associated with the medication to be restocked, which barcode corresponds to an identification number which serves as the indication of the medication.

19. The method of claim 1, wherein the RFID tag is associated with a unique identification associated with the medication housed by the corresponding medication bin.

20. The method of claim 19, wherein the unique identification comprises at least one of a National Drug Code (NDC) or a Drug Identification Number (DIN).

21. A method of prioritizing a plurality of antennas with respect to at least one of a plurality of radio frequency identification (RFID) tags, said method comprising:
associating the plurality of RFID tags with a corresponding plurality of medication bins, said plurality of medication bins configured to respectively house a corresponding plurality of medications;
determining a signal strength associated with respective antennas of the plurality of antennas with respect to at least one of the plurality of RFID tags; and
maintaining an ordering of the plurality of antennas for each RFID tag, wherein the ordering corresponds to the signal strength of respective antennas for the respective RFID tag.

22. The method of claim 21, wherein determining a signal strength associated with respective antennas comprises, for at least one of the plurality of RFID tags:
transmitting an inquiry to the RFID tag via each of the plurality of antennas;
receiving, in response, a confirmation signal from the RFID tag via at least some of the plurality of antennas; and
determining a signal strength associated with each of the plurality of antennas, based at least in part on a signal strength associated with each of the confirmation signals.

23. An inventory management system comprising:
a plurality of medication bins configured to respectively house a corresponding plurality of medications;
a plurality of radio frequency identification (RFID) tags associated with the plurality of medication bins, respective RFID tags associated with a unique identification corresponding to the medication housed by the associated medication bin, respective RFID tags comprising a notification device; and
a software application adapted to operate on a processing element and configured to generate and transmit a signal to at least one of the plurality of RFID tags, wherein, upon receiving the signal, the RFID tag is configured to activate the corresponding notification device.

24. The system of claim 23, wherein the notification device comprises a light emitting diode (LED).

25. The system of claim 23, wherein the notification device comprises a liquid crystal display (LCD).

26. The system of claim 23, wherein the application is further configured to receive a selection of an order to process, said order comprising a medication to be dispensed, such that transmitting a signal to at least one of the plurality of RFID tags comprises transmitting the signal to the RFID tag associated with the medication bin configured to house the medication to be dispensed.

27. The system of claim 23, wherein the application is further configured to receive an indication of a medication to be restocked, such that transmitting a signal to at least one of the plurality of RFID tags comprises transmitting the signal to the RFID tag associated with the medication bin configured to house the medication to be restocked.

28. The system of claim 27 further comprising:
a bar code scanner configured to scan a barcode associated with a medication, such that the indication of a medication to be restocked comprises an identification number based upon the barcode captured by the barcode scanner and associated with the medication to be restocked.

29. The system of claim 28, wherein respective medication bins further comprise a barcode identifying the medication housed by the corresponding medication bin, and wherein the software application is further configured to:
receive an identification of a medication from a barcode associated with a medication bin and captured by the barcode scanner;
determine whether the identification matches the medication to be restocked; and
generate an error message where the identification does not match the medication to be restocked.

30. The system of claim 26, wherein the application is further configured to:
receive an identification of a medication retrieved from a medication bin;
compare the identification with the medication of the order received to process; and
generate an error message where the indication does not match the medication of the order received.

31. The system of claim 30 further comprising:
a barcode scanner configured to scan a barcode associated with a medication, such that the identification of a medication retrieved comprises an identification number based upon a barcode associated with the medication retrieved and captured by the barcode scanner.

32. The system of claim 23 further comprising:
a plurality of antennas, wherein respective antennas are configured to communicate with at least one of the plurality of RFID tags, and wherein the application is further configured to determine via which of the plurality of antennas to transmit the signal to the at least one RFID tag.

33. The system of claim 32, wherein, in order to determine via which of the plurality of antennas to transmit the signal to the at least one RFID tag, the application is further configured to:
transmit an inquiry to the RFID tag via each of the plurality of antennas;
receive, in response, a confirmation signal from the RFID tag via at least some of the plurality of antennas; and
determine a signal strength associated with each of the plurality of antennas, based at least in part on a signal strength associated with each of the confirmation signals, wherein the antenna having the strongest signal strength comprises the antenna via which the signal is transmitted.

34. A shelving unit comprising:
a plurality of medication bins configured to respectively house a corresponding plurality of medications; and
a plurality of radio frequency identification (RFID) tags associated with the plurality of medication bins, respective RFID tags associated with a unique identification corresponding to the medication housed by the associated medication bin, respective RFID tags comprising a notification device, wherein respective RFID tags are configured to receive a signal and, upon receipt of the signal, activate the corresponding notification device.

35. The shelving unit of claim 34, wherein the notification device comprises a light emitting diode (LED).

36. The shelving unit of claim 34, wherein the notification device comprises a liquid crystal display (LCD).

37. A computer program product for managing inventory, wherein the computer program product comprises at least one computer-readable storage medium having computer-readable program code portions stored therein, the computer-readable program code portions comprising:
a first executable portion for receiving an indication of a medication; and
a second executable portion for generating and transmitting a signal to a radio frequency identification (RFID) tag associated with a medication bin configured to house the medication, wherein, upon receiving the signal, the RFID tag is configured to activate a notification device associated with the RFID tag.

38. The computer program product of claim 37, wherein transmitting a signal to a radio frequency identification (RFID) tag comprises transmitting the signal via at least one of a plurality of antennas.

39. The computer program product of claim 38 further comprising:
a third executable portion for determining via which of the plurality of antennas to transmit the signal.

40. The computer program product of claim 39, wherein, in order to determine via which of the plurality of antennas to transmit the signal, the computer-readable program code portions further comprise:
a fourth executable portion for transmitting an inquiry to the RFID tag via each of the plurality of antennas;
a fifth executable portion for receiving, in response, a confirmation signal from the RFID tag via at least some of the plurality of antennas; and
a sixth executable portion for determining a signal strength associated with each of the plurality of antennas, based at least in part on a signal strength associated with each of the confirmation signals, wherein the antenna having the strongest signal strength comprises the antenna via which the signal is transmitted.

41. The computer program product of claim 40, wherein the computer-readable program code portions further comprise:
a seventh executable portion for maintaining an ordering of the plurality of antennas for each RFID tag, wherein the ordering corresponds to the signal strength associated with respective antennas for the respective RFID tag.

42. The computer program product of claim 39, wherein the step of determining via which of the plurality of antennas to transmit the signal is repeated for each of a plurality of RFID tags corresponding with a plurality of medication bins.
